(19) United States
(12) Patent Application Publication
(10) Pub. No.: US 2023/0414837 A1
(43) Pub. Date: Dec. 28, 2023

(54) Polypeptide Polymer-Doped Bone Marrow Cavity Filler and Use Thereof in Treatment of Osteomyelitis

(71) Applicants: EAST CHINA UNIVERSITY OF SCIENCE AND TECHNOLOGY, Shanghai (CN); SHANGHAI FIRST PEOPLE'S HOSPITAL, Shanghai (CN)

(72) Inventors: Runhui Liu, Shanghai (CN); Haodong Lin, Shanghai (CN); Yueming Wu, Shanghai (CN)

(73) Assignees: EAST CHINA UNIVERSITY OF SCIENCE AND TECHNOLOGY, Shanghai (CN); SHANGHAI FIRST PEOPLE'S HOSPITAL, Shanghai (CN)

(21) Appl. No.: 18/005,114

(22) PCT Filed: Nov. 16, 2021
(86) PCT No.: PCT/CN2021/130974

§ 371 Date: Jan. 11, 2023

(30) Foreign Application Priority Data
Feb. 5, 2021 (CN) 202110162718.0

(51) Int. Cl. A61L 27/48; A61L 27/56

(52) U.S. Cl. CPC A61L 27/48; A61L 27/56; A61L 2300/404; A61L 2430/02

(57) ABSTRACT

Disclosed are a polypeptide polymer-doped bone marrow cavity filler and the use thereof in the treatment of osteomyelitis, wherein the polypeptide polymer is used for being doped in a bone marrow cavity filler or preparing a bone marrow cavity filling antibacterial material for treating osteomyelitis, has efficient antibacterial activity on common staphylococcus aureus, etc., in osteomyelitis, is not easy to induce bacteria to generate drug resistance, has good biocompatibility in environments such as bone marrow and blood, has good stability, and still keeps activity after forming heat release and even the autoclaving of bone cement.

### TECHNICAL FIELD

The invention relates to the field of the prevention and treatment of osteomyelitis disease, in particular to a bone cement doped with a polypeptide polymer or a polypeptide mimic as an antibiotic instead of an antibacterial agent.

### BACKGROUND TECHNIQUE

Osteomyelitis is a relatively common bone disease, involving infection and destruction of bones. If timely treatment is not taken, it will cause great harm to the human body, and it is easy to cause corresponding lesions in other parts of the body. Chronic osteomyelitis is a continuation of acute suppurative osteomyelitis. Symptoms are usually localized, and are often stubborn and refractory. The inflammation recurs and cannot be cured even after several years or more than a decade. Antibiotic therapy is usually used clinically, but with the serious abuse of antibiotics, the emergence of drug-resistant bacteria has brought great challenges to the treatment of osteomyelitis. Therefore, there is an urgent need in the art to develop a type of bone marrow cavity filler with high antibacterial activity, good biocompatibility, simple preparation process and low cost for the anti-infection treatment of osteomyelitis.

### SUMMARY OF THE INVENTION

The object of the present invention is to provide a bone marrow cavity filler that can be used for the anti-infection treatment of osteomyelitis.

The first aspect of the present invention provides a use of a polypeptide polymer for doping a bone marrow cavity filler; or for the preparation of an antibacterial material for filling bone marrow cavity for treating osteomyelitis.

Bone marrow cavity filler doped with polypeptide polymers is used to treat osteomyelitis and used as an alternative to antibiotics to kill or inhibit the growth of pathogenic bacteria.

In another preferred embodiment, the polypeptide polymer is resistant to high temperature during use.

In another preferred embodiment, the polypeptide polymer is resistant to protease during use.

In another preferred embodiment, the polypeptide polymer is not easy to bacteria to develop drug resistance during use.

In another preferred embodiment, the osteomyelitis is chronic osteomyelitis or acute osteomyelitis.

In another preferred embodiment, the osteomyelitis occurs in the metaphysis of long bone such as the tibia or femur, diabetic foot, penetrating bone injury, and the like.

In another preferred embodiment, the bone marrow cavity filler is polymethacrylic acid (PMMA) bone cement, calcium phosphate bone cement (CPC), calcium sulfate bone cement, bioglass, hydroxyapatite, bioceramic, or gelatin sponge. In another preferred embodiment, the doping includes powder doping or solution doping.

In another preferred embodiment, the doping dose of the polypeptide polymer is 1 wt %-20 wt % or 1 wt %-40 wt % based on the weight of the filler.

In another preferred embodiment, the doping dose of the polypeptide polymer is 5 wt %-15 wt % based on the weight of the filler.

In another preferred embodiment, the pathogenic bacterium of the osteomyelitis is aerobic or anaerobic bacterium, mycobacteria and/or fungi, and is selected from Staphylococcus aureus, hemolytic streptococcus, staphylococcus albus, pneumococcus, Escherichia coli, Pseudomonas aeruginosa or a combination thereof.

In another preferred embodiment, the polypeptide polymer is a homopolymer comprising a lysine residue or a copolymer comprising a lysine residue and a benzyl glutamate residue,

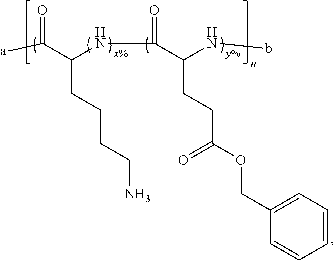

the configuration is L, D or DL;

the chain length n is 1-1000, x % is 100%-30%, y % is 0-70%;

the terminal groups a, and b are each independently H, amino, hydroxyl, C1-C15 alkyl, C1-C15 alkyleneamino, C6-C15 aryl, C2-C15 alkenyl, C2-C15 alkynyl, C1-C15 alkylenehydroxyl, C1-C15 alkylene aldehyde group, C1-C15 alkylene ester group, thio-C1-C15 alkylene ester group, 5-15 membered heteroaryl, or 5-12 membered heterocyclyl.

In another preferred embodiment, the terminal groups a, and b are each independently H, amino, hydroxyl, C1-C10 alkyl, C1-C10 alkyleneamino, C6-C10 aryl, C2-C10 alkenyl, C2-C10 alkynyl, C1-C10 alkylenehydroxyl, C1-C10 alkylene aldehyde group, C1-C10 alkylene ester group, thio-C1-C10 alkylene ester group, 5-8 membered heteroaryl, or 5-8 membered heterocyclyl.

In another preferred embodiment, the terminal groups a, and b are each independently H, amino, hydroxyl, C1-C6 alkyl, C1-C6 alkyleneamino, C6-C6 aryl, C2-C6 alkenyl, C2-C6 alkynyl, C1-C6 alkylenehydroxyl, C1-C6 alkylene aldehyde group, C1-C6 alkylene ester group, thio-C1-C6 alkylene ester group, 5-7 membered heteroaryl, or 5-7 membered heterocyclyl.

In another preferred embodiment, the terminal groups a, and b are each independently H, amino, hydroxyl, C1-C4 alkyl, C1-C4 alkyleneamino, C4-C4 aryl, C2-C4 alkenyl, C2-C4 alkynyl, C1-C4 alkylenehydroxyl, C1-C4 alkylene aldehyde group, C1-C4 alkylene ester group, thio-C1-C4 alkylene ester group, 6 membered heteroaryl, or 6 membered heterocyclyl.

In another preferred embodiment, the polypeptide polymer is the polymer prepared in the Examples.

In another preferred embodiment, the polypeptide polymer has good biocompatibility, has no obvious hemolytic activity on human red blood cells, mouse red blood cells and the like; and shows no obvious cytotoxicity to mammalian cells such as mouse embryonic fibroblasts, African monkey kidney cells, human umbilical vein endothelial cells, and canine kidney cells.

The second aspect of the present invention provides an antibacterial material for filling bone marrow cavity, comprising a polypeptide polymer and a bone marrow cavity filler.

In another preferred embodiment, the antibacterial material for filling bone marrow cavity is an antibacterial material for filling bone marrow cavity for treating osteomyelitis.

In another preferred embodiment, the bone marrow cavity filler is polymethacrylic acid (PMMA) bone cement, calcium phosphate bone cement (CPC), calcium sulfate bone cement, bioglass, hydroxyapatite, bioceramic, or gelatin sponge.

In another preferred embodiment, the polypeptide polymer is a homopolymer comprising a lysine residue or a copolymer comprising a lysine residue and a benzyl glutamate residue,

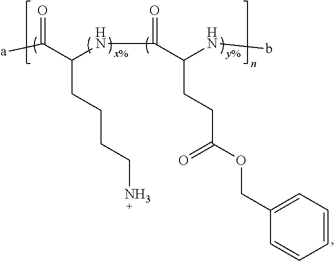

the configuration is L, D or DL;

the chain length n is 1-1000, x % is 100%-30%, y % is 0-70%;

the terminal groups a, and b are each independently H, amino, hydroxyl, C1-C15 alkyl, C1-C15 alkyleneamino, C6-C15 aryl, C2-C15 alkenyl, C2-C15 alkynyl, C1-C15 alkylenehydroxyl, C1-C15 alkylene aldehyde group, C1-C15 alkylene ester group, thio-C1-C15 alkylene ester group, 5-15-membered heteroaryl, or 5-12-membered heterocyclyl.

In another preferred embodiment, the polypeptide polymer is the polymer prepared in the Examples.

In another preferred embodiment, the weight ratio of the polypeptide polymer to the bone marrow cavity filler is 1-40:99-60, 1-20:99-80, 5-15:95-85, or 8-12: 92-88.

The bone marrow cavity filler doped with the polypeptide polymer according to the present invention is used for the treatment of chronic osteomyelitis and acute osteomyelitis, has high antibacterial activity against Staphylococcus aureus commonly seen in osteomyelitis and is not easy to induce bacteria to develop drug resistance, and has good biocompatibility in bone marrow, blood and other environments. Moreover, the polypeptide polymer has good stability, and remains active even after heat release of bone cement forming and even autoclave sterilization.

It should be understood that within the scope of the present invention, the above mentioned technical features of the present invention and the technical features specifically described in the following (eg, the examples) can be combined with each other to form new or preferred technical solution. Each feature disclosed in the specification may be replaced by any alternative feature serving the same, equivalent or similar purpose. Due to space limitations, it is not repeated herein.

### DESCRIPTION OF DRAWINGS

FIG. 1 shows the infrared, contact angle and XPS test results of polypeptide polymer PMMA bone cement.

FIG. 2 shows the result of the strength test of the polypeptide polymer PMMA bone cement.

FIG. 3 shows the result of the antibacterial activity test of the polypeptide polymer solution.

FIG. 4 is a graph of the antibacterial effect.

FIG. 5 shows the result of the stability test of the polypeptide polymer.

FIG. 6 shows the result of a hemolytic activity test on red blood cells.

FIG. 7 shows the result of live/dead cell staining microscopy.

FIG. 8 shows the result of MTT quantitative test.

FIG. 9 shows the statistical result of white blood cell count in the blood routine test.

FIG. 10 is a graph of the X-ray detection result.

FIG. 11 is a photograph of the upper end of the tibia.

FIG. 12 shows the result of bacterial weighing homogenate plating of bone marrow tissue.

FIG. 13 shows the result of bacterial count in bone tissue and bone marrow tissue.

FIG. 14 is a staining diagram of liver and kidney tissue sections.

FIG. 15 is a Gram-stained image of bone tissue sections.

FIG. 16 is a immunofluorescence image of the section.

FIG. 17 shows the treatment result of polypeptide polymer gelatin sponge on osteomyelitis.

### DETAILED DESCRIPTION

The following illustrates that the antibacterial polymer prepared in the invention can be used for the anti-infection treatment of osteomyelitis in combination with the specific examples.

### EXAMPLE 1

### Preparation of Polypeptide Polymer From N-ϵ-Tert-Butoxycarbonyl-DL-Lysine-N-Carboxyanhydride and 5-Benzyl L-Glutamate-N-Carboxyanhydride Initiated by Lithium Hexamethyldisilazide (LiHMDS)

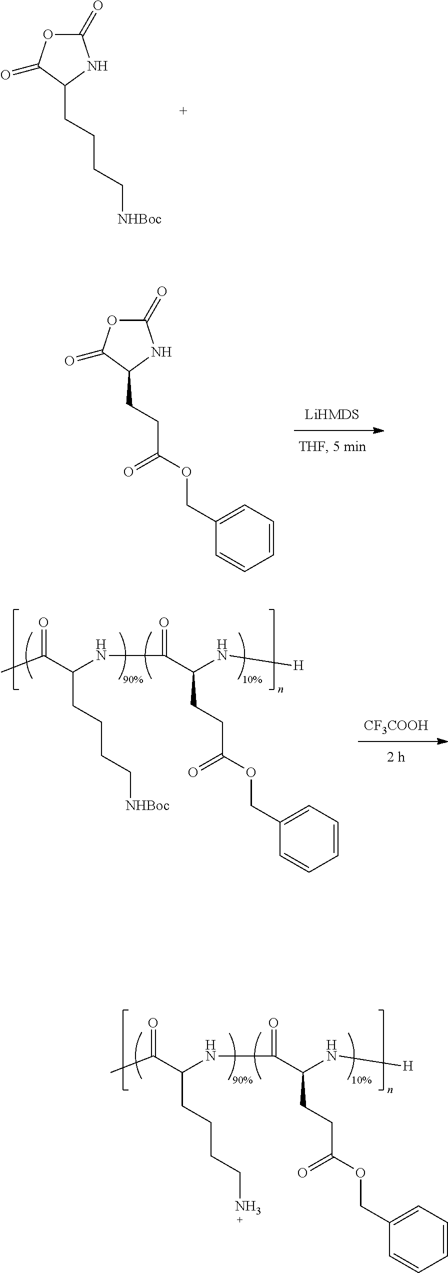

N-ϵ-tert-butoxycarbonyl-DL-lysine-N-carboxyanhydride and 5-benzyl L-glutamate-N-carboxyanhydride were weighed and tetrahydrofuran was used as solvent. 9 equivalents of N-ϵ-tert-butoxycarbonyl-DL-lysine-N-carboxyanhydride and 1 equivalent of 5-benzyl L-glutamate-N-carboxyanhydride were mixed and stirred with a magnetic stir bar. One-fifth of the total monomer equivalent of the initiator lithium hexamethyldisilazide was weighed to prepare a solution and added quickly. The reaction was carried out at room temperature for 5 minutes. A large amount of petroleum ether was added to precipitate a white flocculent precipitate, which was collected by filtration to obtain a protected polymer (6 g, n is 27). After adding trifluoroacetic acid to the protected polymer, it was shaken for 6 hours to remove the protective group. After adding iced methyl tert-butyl ether to precipitate a white precipitate, it was collected by filtration. The sample was dissolved in ultrapure water, and finally lyophilized to obtain the deprotected of polypeptide polymer.

### EXAMPLE 2

### Preparation of Bone Marrow Cavity Filler

### 2.1 Polypeptide Polymer PMMA Bone Cement

PMMA bone cement was composed of polymethyl methacrylate (powder) and monomer methyl acrylate (liquid), the powder included PMMA, styrene and initiator, etc.; the liquid was methyl methacrylate (MMA) and accelerators, etc. The bone cement powder and the liquid were prepared for use in a ratio of 2:1 (g:mL), and the polypeptide polymer (accounting for 8 wt % of the bone cement powder) prepared in Example 1 was pre-dissolved in a small amount of DMSO to prepare a 0.4M polymer solution, which was added to the pre-prepared liquid and mixed well. The liquid containing the polymer solution was added to the pre-prepared bone cement powder, stirred and mixed for 2 minutes, and then transferred to the mold. The mixture was compacted with a steel plate for 15 minutes and then taken out from the mold. A cylindrical bone cement with a diameter and thickness of about 3 mm was prepared.

### 2.2 Polypeptide Polymer Gelatin Sponge

The gelatin sponge was cut into a rectangle of 2*1 cm, and the excess cross-linking agent was removed by repeated washing with ultrapure water. After the last washing, 0.5 ml aqueous solution of the polypeptide polymer (15 mg) prepared in Example 1 was added for adsorption, and the obtained gelatin sponge was frozen in liquid nitrogen and then freeze-dried in a freeze-drying machine to obtain the gelatin sponge adsorbing the polypeptide polymer.

### EXAMPLE 3

### Characterization of Bone Marrow Cavity Filler

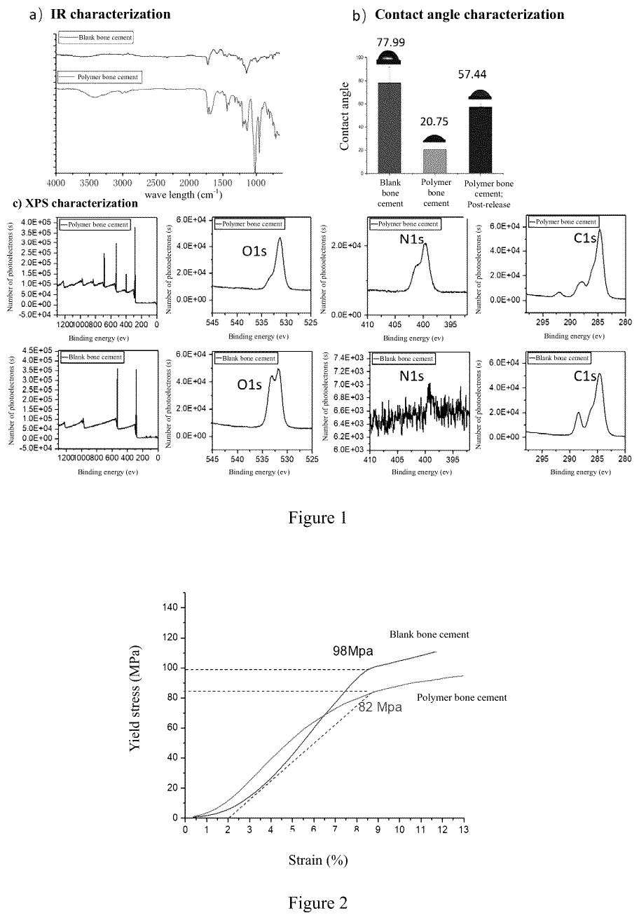

In order to prove the successful preparation of the bone marrow cavity filler and the successful incorporation of the polypeptide polymer, the polypeptide polymer PMMA bone cement prepared in Example 2 was subjected to infrared, contact angle and XPS tests. The results were shown in FIG. 1. The infrared spectrum of polypeptide bone cement showed the characteristic peaks of the polymer. The result of contact angle characterization showed that the incorporation of polymer changed the contact angle obviously. The characteristic peaks of N and F were contained in the XPS spectrum. Therefore, all tests demonstrated successful incorporation of the polypeptide polymer.

### EXAMPLE 4

### Compressive Strength Test of Polypeptide Polymer PMMA Bone Cement

The compressive strength of polypeptide polymer PMMA bone cement (3 mm diameter and 3 mm thickness) was measured using a universal tensile machine, and the samples were loaded and subjected to radial compression at a rate of 20 mm/min. Each group having 5 cylinders was tested and the mean was calculated. The stress-strain curve of the representative test was shown in FIG. 2. The intersection point was obtained by taking 2% strain as the abscissa and drawing a parallel line and taken as the maximum compressive strength of the sample. The test results showed that both the polypeptide polymer PMMA bone cement and the blank PMMA bone cement exceeded the minimum requirement of 70 MPa required by the national standard.

EXAMPLE 5

### Antimicrobial Activity Test of Polypeptide Polymer Solution

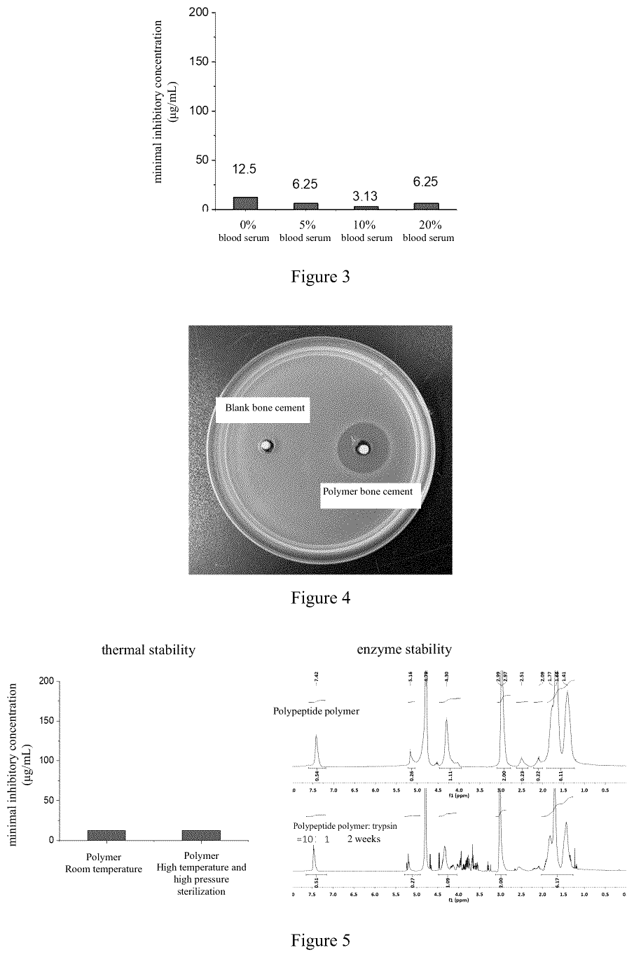

In order to characterize the actual antibacterial activity in the blood environment of osteomyelitis, the minimum inhibitory concentration (MIC) test was performed by adding different proportions of fetal bovine serum (FBS). First, the bacteria were cultivated in LB medium for 10 hours in a shaker with a suitable strain growth temperature of 37° C. Then the mature bacteria were transferred to the centrifuge and centrifuged at 4000 rpm for 5 minutes. The supernatant was removed and the bacteria at the bottom were dispersed with a small amount of MH medium. The OD value was measured on the microplate reader, and the bacterial solution was diluted to 2×10⁵ CFU/mL according to the OD value. 10 μL of the polypeptide polymer to be tested at a concentration of 4 mg/mL was added to the first row of the 96-well plate, then 90 μL of culture medium was added and mixed. 50 μL was taken and diluted step by step from the second row to the eighth row. 50 μL of bacterial solution was added to each well. The medium was used as a negative control, and the bacterial solution was used as a positive control. The MICs of medium mixed with 5%, 10% and 20% FBS were compared by adding different concentrations of serum to the culture medium and diluting them with bacterial solution, so that the serum concentrations in the final test were 5%, 10%, and 20%. After cultured in an incubator at 37° C. for 9 hours, the 96-well plate was placed on a microplate reader and read at a wavelength of 600 nm. Finally, the calculation was performed according to the formula: bacterial growth rate %=(OD^{Polymer}−OD^{blank})/(OD^{control}−OD^{blank})×100%, and each sample was tested in duplicate in the antibacterial activity test. The obtained MIC was shown in FIG. 3. The MIC value of the polymer was reduced under the condition of serum, and the activity was increased. The antibacterial activity of the polymer with 10% serum was increased by 4 times, which proved that the polypeptide polymer had excellent antibacterial activity and did not inactivate in the presence of serum and the activity was improved.

### EXAMPLE 6

### Antibacterial Activity Test of Polypeptide Polymer PMMA Bone Cement

Antibacterial activity was shown by an inhibition zone. First, the bacteria were cultivated in LB medium for 10 hours in a shaker with a suitable strain growth temperature of 37° C. Then the mature bacteria were transferred to the centrifuge and centrifuged at 4000 rpm for 5 minutes. The supernatant was removed and the bacteria at the bottom were dispersed with a small amount of MH medium. The OD value was measured on the microplate reader, and the bacterial solution was diluted to 1×10⁸ CFU/mL according to the OD value for later use. MH solid medium was prepared, in which the mass percentage of agarose replacing agar was 1.5%. The culture medium was sterilized by autoclaving, and after the temperature gradually dropped to 40-50° C., the prepared bacterial solution was added dropwise. The ratio of bacterial solution to medium was 1:99 and the concentration of bacterial solution after mixing and shaking was 1×10⁶ CFU/mL. 20 mL of the culture medium solution with the bacterial solution was poured into a petri dish with a size of 90×15 mm. After the solid medium was solidified, holes were punched by using a sterilized hole puncher with a diameter of 6 mm. The height of the holes was 4-5 mm. 80 μL of PBS was added dropwise to the hole as a solvent, followed by the addition of polypeptide polymer bone cement and blank bone cement to serve as the experimental group and the control group. The petri dishes were placed in a refrigerator at 4° C. for 2 hours to allow pre-diffusion of the drug. After 2 hours, the petri dishes were placed in a 37° C. incubator for culture. The inhibition zone was observed after 24 hours, and the diameter of the inhibition zone was measured using the cross method and recorded. The result of the inhibition zone was shown in FIG. 4, which proved that the polypeptide polymer PMMA bone cement had a significant bacteriostatic effect.

### EXAMPLE 7

### Stability Test of Polypeptide Polymer

The polypeptide polymer in Example 1 was selected for thermal stability and enzyme stability tests. The thermal stability test method was as follows. The polypeptide polymer was weight into a glass bottle. The bottle cap was unscrewed and the bottle was placed in an autoclave. The pressure was increased and the temperature was raised to 120° C. for 30 minutes. Then the bottle was taken out. The peptide polymer treated above was compared with the polymer stored at room temperature without treatment (polymer R.T.). The minimum inhibitory concentration (MIC) for Staphylococcus aureus was tested. FIG. 5 showed that the MIC value remained unchanged, proving that the peptide polymer had thermal stability.

The enzyme stability test method was as follows. After the polypeptide polymer was tested by NMR in advance, trypsin in a weight ratio of 10:1 was added and dissolved in heavy water with PBS for NMR test. The NMR spectrum in FIG. 5 showed that the polymer was not degraded after being placed in the buffer system for two weeks, which proved that the polypeptide polymer had enzyme stability.

### EXAMPLE 8

### Hemolytic Activity Test of Polypeptide Polymer PMMA Bone Cement on Red Blood Cells

The polypeptide polymer PMMA bone cement of Example 2 and the blank PMMA bone cement were used to test the hemolytic activity on red blood cells. The polymer bone cement group and the blank bone cement group were pre-soaked with 0.5 mL Tris buffered saline (TBS) for 24 h before the hemolytic activity test. Fresh human blood provided by volunteers was stored at 4° C. until use. A sufficient amount of human blood was taken for use and diluted with appropriate amount of TBS, and centrifuged at 4000 rpm for 3 minutes on a centrifuge. The supernatant was removed. The red blood cells at the bottom was shaken with TBS and then centrifuged. After the above operations were repeated for 3 times, TBS was added to dilute the red blood cells to 5% for use.

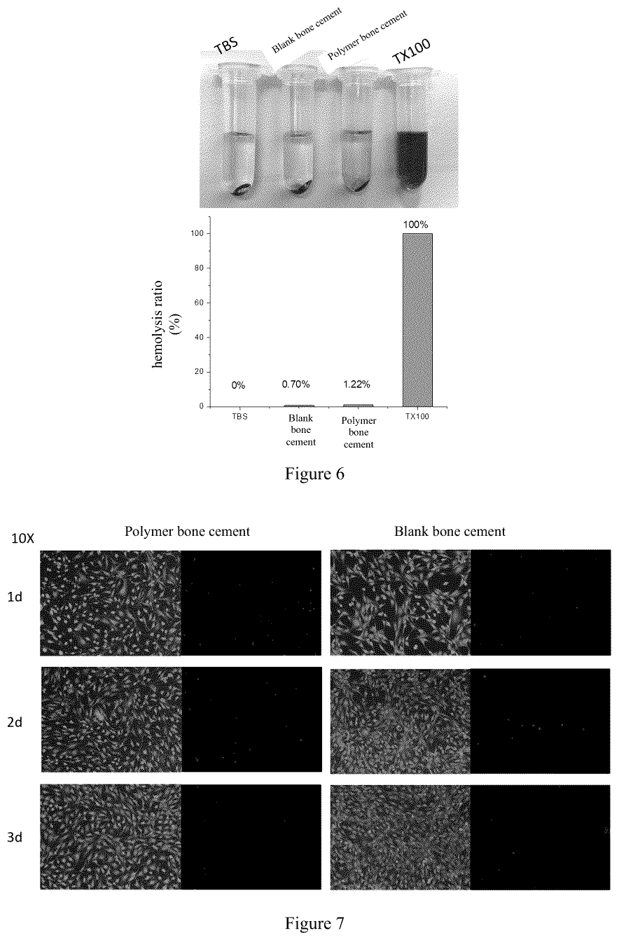

0.5 mL of 5% red blood cell diluent was added with the TBS soaking solution of 0.5 mL polymer bone cement group and TBS soaking solution of blank bone cement group respectively. 0.1% polyethylene glycol octyl phenyl ether (TX100) was used as a positive control. Pure TBS was used as a negative control. After incubating at 37° C. for 1 hour, the mixture was centrifuged at 3700 rpm for 5 minutes. After taking pictures, 100 μL was drawn from each tube to a new 96-well plate, and read on a microplate reader with a wavelength of 405 nm. Finally, the value was calculated according to the formula: hemolysis rate %=(OD^{experimental group}−OD^{TBS negative control})/(OD^{TX100 positive control}−OD^{TBS negative control})×100%. Each sample was tested in duplicate in the hemolytic activity assay. The experimental results were shown in FIG. 6, showing that the polypeptide polymer bone cement and blank bone cement had no obvious hemolytic activity on red blood cells, which proved that they had good biocompatibility with red blood cells.

### EXAMPLE 9

### Cytotoxicity Test of Polypeptide Polymer Bone Cement in Mammalian Cells

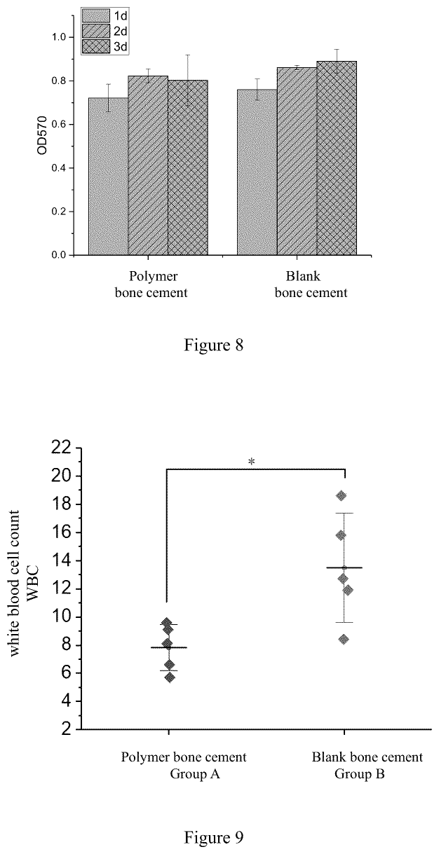

The polypeptide polymer bone cement of Example 2 and blank bone cement were used to test the cytotoxicity to mouse fibroblasts NIH3T3. The polymer bone cement group and the blank bone cement group were pre-soaked in 5 mL of DMEM medium for 24 hours, respectively, and the leaching solution was taken for cytotoxicity test. The monolayer cells were first digested with trypsin, collected after the cells fell off, and centrifuged at 1200 rpm for 4 minutes in a centrifuge to sediment the cells. The supernatant was discarded, and the cells were resuspended in culture medium for counting. The cells were diluted with 24 h leaching solution to 8×10⁴ cells/mL, and 100 μL was transferred to each well of a 96-well plate. Then the 96-well plate was placed in a 37° C., 5% CO₂ incubator for incubation for a period of time. Live/dead cells were stained and observed by microscopy. The method of MTT quantitative test was as follows. The medium in the well plate was removed and 100 μL of thiazolyl blue (MTT) dye (0.5 mg/mL) was added. The plate was placed in an incubator for 4 hours for staining, and then the MTT dye was removed and 150 μL of dimethyl sulfoxide was added. The 96-well plate was put on a shaker for 15 minutes to mix well, then put it into a microplate reader and read at 570 nm. Each sample was tested in triplicate in the cytotoxicity test. The micrographs on day 1, day 2 and day 3 were shown in FIG. 7, and the MTT quantitative test results on day 1, day 2 and day 3 were shown in FIG. 8, indicating that the tested polypeptide polymer bone cement had no obvious cytotoxicity on mammalian cells compared with the blank bone cement.

### EXAMPLE 10

### Therapeutic Effect of Polypeptide Polymer PMMA Bone Cement on Chronic Osteomyelitis in Rabbits Infected by Bacteria

Establishment of osteomyelitis model: New Zealand rabbits, male, with a body weight of 2.5 kg-3.0 kg were raised. Tibial osteomyelitis model was established. The rabbits were anesthetized by intravenous injection of 10% chloral hydrate (2.5 mL/kg) at the ear margin and fixed in the supine position on the operating table. The skin at the right tibia was prepared, routinely disinfected and draped. The skin was cut longitudinally at the front medial edge of the tibia as the starting point, the muscles and fascia were separated, and after the tibia was exposed, a 1 cm³ defect was made at the upper end of the tibia with a 5 mm Kirschner wire to open the bone marrow cavity. After extracting bone marrow with 1 mL syringe, 0.1 mL methicillin-resistant Staphylococcus aureus MRSA (1×10⁹ CFU/ml) was injected into the marrow cavity. The pore formed by the syringe was sealed with bone wax again to prevent bacterial fluid leakage. Finally, the soft tissues and skin were sutured layer by layer, the incision was covered with povidone iodine sterile gauze, and the animals were raised in a single cage according to the unified standard for 4 weeks.

Evaluation of Osteomyelitis Model: At 4 weeks after surgery, the chronic osteomyelitis model was evaluated. The method was as follows. Before and after modeling, weight and body temperature were measured and recorded. General observation: The wound healing and soft tissue condition of the experimental rabbits were observed for the sinus tract formation and soft tissue swelling. The upper end of the tibia was dissected and the bone destruction, hyperplasia and bone defect healing were generally observed. X-ray findings: 4 weeks after operation, X-ray detection was performed on the surviving rabbits to observe the imaging manifestations of osteomyelitis, and to observe whether there was local sequestrum formation, bone destruction, bone hyperplasia and soft tissue inflammatory mass shadow. The treatment of osteomyelitis was evaluated semi quantitatively with Norden scoring method. Bacterial culture of bone marrow tissue (gold standard): sinus and purulent secretions, bone marrow tissue and bone tissue were taken for bacterial culture.

Surgical treatment of osteomyelitis: The rabbits with successful modeling were randomly divided into 2 groups (n≥6). Group A: control group (simple debridement+implantation of blank bone cement). Group B: polypeptide polymer bone cement group (debridement+implantation of polypeptide polymer bone cement). After anesthesia, the skin of the right tibia was prepared, routinely sterilized and draped, followed by the original incision, the muscle and fascia were incised, the tibia was exposed, and the bone resorption and deformity of the tibial shaft were observed. A large amount of normal saline was used to flush the bone marrow cavity to completely remove inflammatory and necrotic tissues. For those with sinus tracts, the sinus tract was removed, and the bone marrow cavity was flushed until there was no inflammatory tissue. Group A was simply implanted with 10 blank bone cement particles (250 mg PMMA), and group B was implanted with 10 particles of polypeptide PMMA polymer bone cement products (200 mg PMMA+6 wt % polypeptide polymer) into the bone marrow cavity. All were sealed with bone wax, the soft tissue and skin were sutured layer by layer, the incision was covered with sterile gauze sterilized with povidone iodine, and the animals were reared in a single cage according to the unified standard for 2 weeks.

Treatment results of osteomyelitis: 2 weeks after the second operation, body weights were measured and recorded. Blood was drawn from the edge of the ear for routine blood tests. General observation: The wound healing and soft tissue condition of the experimental rabbits were observed to see whether there was local redness and swelling, sinus or purulent secretions and to see if they were better than before. If there was purulent secretion, the secretion was taken for bacterial culture. The upper end of the tibia was dissected and the bone destruction, hyperplasia and bone defect healing were generally observed. X-ray findings: 6 weeks after the first operation, X-ray detection was performed on the surviving rabbits, and the imaging manifestations of osteomyelitis were observed to see if there was the local sequestrum formation, bone destruction, bone hyperplasia or soft tissue inflammatory mass shadow. Bone marrow tissue bacterial culture: 2 weeks after the operation, the rabbits were sacrificed and part of the bone tissue 5-10 mm around the defect, left liver, left kidney and other tissues were taken for fixation for subsequent histological staining. Bone tissue, bone marrow tissue 5-10 mm around the defect, left liver and left kidney and other tissues were weighed and homogenized and then coated for bacterial culture. The specific operations were as follows.

The specific weighing steps are as follows. The tissue was weighted in a 2 mL centrifuge tube and a homogenate (a PBS solution with a volume fraction of 0.1% TX100) and a large steel ball were added at 4.5 μL/mg (100-200 mg). The 2 mL centrifuge tube for homogenization was sterilized in advance. The equipment for cutting tissue was sterilized. After cutting, each tissue was wiped with 75% alcohol and dried before use. The required material quality was estimated according to the volume of liquid added and the volume of large steel ball. Bone tissue was crushed into small pieces with a rongeur.

The specific homogenization steps were as follows: 60 Hz homogenate was used for 120 seconds except for bone tissue for 5 minutes. Homogenized nylon centrifugal pipe rack was sprayed with alcohol for air drying before use.

The specific dilution steps were as follows: the homogenized centrifuge tube settled naturally for 1 min. 100-200 μL of the stock solution close to the solid was sucked out and put into a new 1.5 mL sterile centrifuge tube, and then diluted 10 times with PBS to the required concentration after mixing.

The specific coating steps were as follows. 20 μL was taken and used to coat. Before coating, the mixture in centrifuge tube was mixed uniformly and then used to coat. The pipette tip should not touch the agar plate. The coating was as close to the edge as possible. The plate was reversed all the time. The bacterial plates were counted 12 hours later.

FIG. 9 showed the white blood cell count statistics in the blood routine test. There was a significant difference between the blank (group A) and the polymer bone cement group (group B), indicating that the infection was controlled after aggressive anti-infection treatment with polypeptide polymer PMMA bone cement, and the white blood cell count was normal.

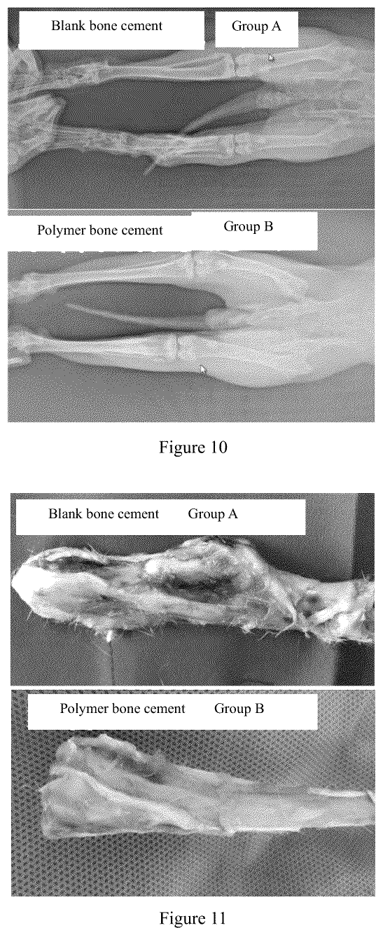

6 weeks after the first operation and 2 weeks after the second operation, X-ray detection was performed on the surviving rabbits. As shown in FIG. 10, in group A, i.e., the blank bone cement group, osteomyelitis was serious with bone hyperplasia, irregular bone cavity, dead bone formation, and pathological fracture. In group B, polypeptide polymer PMMA bone cement was used. In the polymer bone cement group, bone destruction was less and bone defects were gradually healed.

The upper end of the tibia was dissected for general observation, as shown in FIG. 11. Sequestrum and pathological fractures were seen in group A(blank bone cement group), and there was no sinus tract and purulent secretions in group B (polymer bone cement group), and the bone was normal.

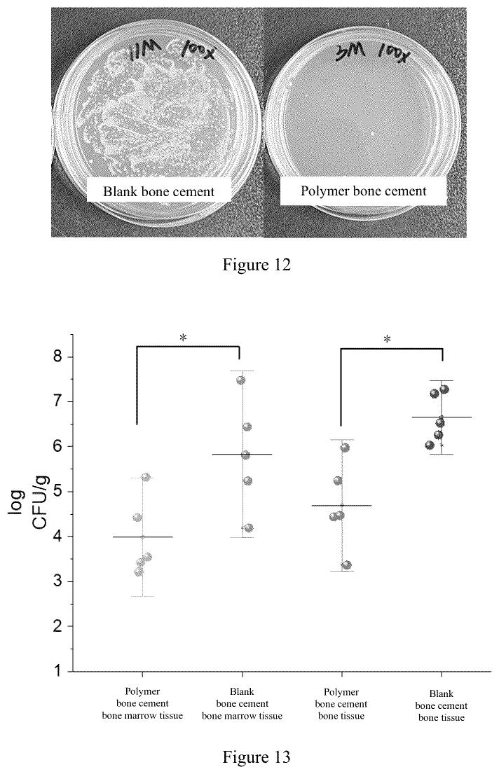

The bacteria in bone marrow tissue were weighed, homogenized and coated, as shown in FIG. 12, there was a significant difference between the blank bone cement group and the polymer bone cement group. After 100-fold dilution, trace colonies grew in the polymer bone cement group, and a large number of colonies grew in the blank bone cement group after 100-fold dilution.

The bacterial counts in bone tissue and bone marrow tissue were shown in FIG. 13. There was a significant difference between the blank bone cement group and the polymer bone cement group. The bacterial counts in the bone and bone marrow tissue of the polymer bone cement group decreased, indicating that the polymer bone cement group was treated effectively.

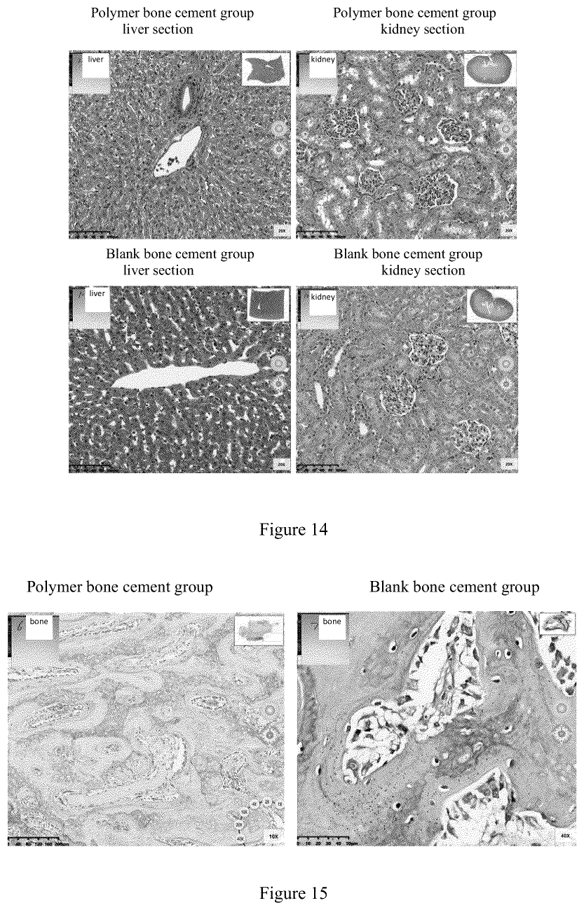

Liver and kidney tissue sections were shown in FIG. 14. Compared with the blank bone cement group, the polymer bone cement group had no toxicity and no obvious tissue damage.

Gram staining of bone tissue sections was shown in FIG. 15. A large number of bacteria aggregated in the blank bone cement group, but there was no large amount of bacteria in the polymer bone cement group.

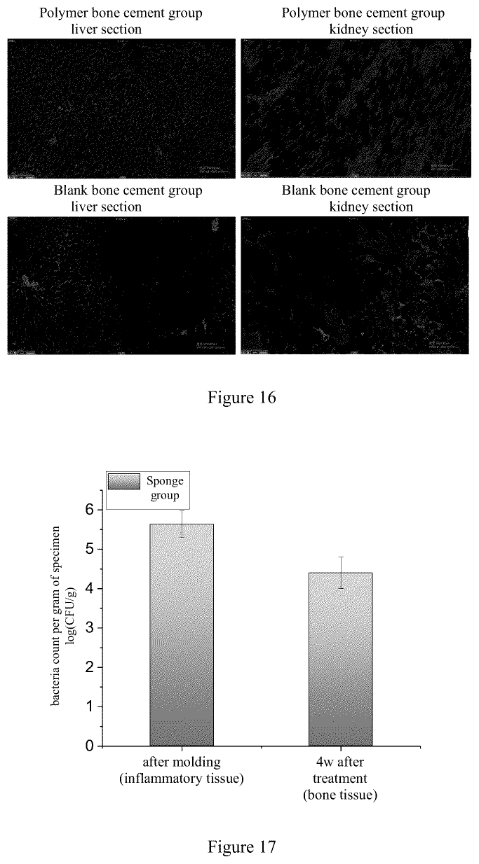

The expression of the macrophage marker CD68 in immunofluorescence sections was shown in FIG. 16. CD68 in the polymer bone cement group and the blank bone cement group were positively expressed, but the polymer bone cement group could attenuate the inflammatory response of the tissue.

### EXAMPLE 11

### Therapeutic Effect of Polypeptide Polymer Gelatin Sponge on Osteomyelitis

After the osteomyelitis model was successfully established by using the method of Example 10, inflammatory tissue was generated. After taking the inflammatory tissue and counting the number of colonies, the treatment was performed according to the surgical treatment method for osteomyelitis in Example 10, except that the polymer bone cement was replaced with polypeptide polymer gelatin sponge.

The treatment results of osteomyelitis were shown in FIG. 17. Bone tissue was taken, weighed, homogenized and coated to calculate the number of colonies. Compared with that after modeling, the number of colonies in the polymer sponge group decreased significantly.

All documents mentioned in the present invention are incorporated by reference in this application, as each document is individually incorporated by reference. In addition, it should be understood that after reading the above-mentioned teaching content of the present invention, those skilled in the art can make various changes or modifications to the present invention, and these equivalent forms also fall within the scope defined by the appended claims of the present application.

1-6. (canceled)

7. An antibacterial material for filling a bone marrow cavity comprising a polypeptide polymer and a bone marrow cavity filler.

8. The antibacterial material according to claim 7, wherein the bone marrow cavity filler is polymethacrylic acid bone cement, calcium phosphate bone cement, calcium sulfate bone cement, bioglass, hydroxyapatite, bioceramic, or gelatin sponge.

9. The antibacterial material according to claim 7, wherein the polypeptide polymer is a homopolymer comprising a lysine residue or a copolymer comprising a lysine residue and a benzyl glutamate residue, 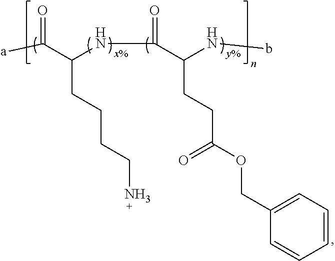
the configuration of the lysine residue or the benzyl glutamate residue is L, D or DL;
the chain length n is 1-1000, x % is 100%-30%, y % is 0-70%; and
the terminal groups a, and b are each independently H, amino, hydroxyl, C1-C15 alkyl, C1-C15 alkyleneamino, C6-C15 aryl, C2-C15 alkenyl, C2-C15 alkynyl, C1-C15 alkylenehydroxyl, C1-C15 alkylene aldehyde group, C1-C15 alkylene ester group, thio-C1-C15 alkylene ester group, 5-15-membered heteroaryl, or 5-12-membered heterocyclyl.

10. The antibacterial material according to claim 7, wherein a weight ratio of the polypeptide polymer and the bone marrow cavity filler is 1-40:99-60.

11. A method for treating osteomyelitis in a subject in need thereof comprising filling a bone marrow cavity of the subject with an effective amount of the antibacterial material of claim 7.

12. The method of claim 11, wherein the subject is suffering from chronic osteomyelitis.

13. The method of claim 11, wherein the subject is suffering from acute osteomyelitis.

14. The method of claim 11, wherein osteomyelitis occurs in metaphysis of a tibia or a femur, diabetic foot, or a penetrating bone injury.

15. The method of claim 11, wherein the antibacterial material is used as an alternative to antibiotics to inhibit grown of pathogenic bacteria.

16. The method of claim 11, wherein osteomyelitis is caused by a pathogenic bacterium or a pathogenic fungus.

17. The method of claim 16, wherein the pathogenic bacterium is selected from the group consisting of: a mycobacterium, Staphylococcus aureus, a hemolytic streptococcus, Staphylococcus albus, a pneumococcus bacterium, Escherichia coli, Pseudomonas aeruginosa, and a combination thereof.

18. The method of claim 11, wherein the antibacterial material is prepared by powder doping or solution doping.
